(19) United States
(12) Patent Application Publication
(10) Pub. No.: US 2003/0219431 A1
(43) Pub. Date: Nov. 27, 2003

(54) Treatment of neuronal and neurological damage associated with spinal cord injury

(75) Inventors: Samn Petti, East Durham, NY (US); Stephen J. Petti, East Durham, NY (US)

Correspondence Address: HESLIN ROTHENBERG FARLEY & MESITI PC, 5 COLUMBIA CIRCLE, ALBANY, NY, 12203, US

(73) Assignee: Empire Pharmaceuticals, Inc., Greenville, NY (US)

(21) Appl. No.: 10/445,596

(22) Filed: May 27, 2003

Related U.S. Application Data
(60) Provisional application No. 60/383,321, filed on May 24, 2002.
(60) Provisional application No. 60/423,957, filed on Nov. 4, 2002.

(51) Int. Cl. A61K 38/48; A61K 38/46

(52) U.S. Cl. 424/94.64; 424/94.65

(57) ABSTRACT

A method for the treatment of spinal cord injury is disclosed. The method involves the administration of a therapeutically effective amount of a defibrinogenating or fibrinolytic agent. In accordance with the principles of the invention, also disclosed are a method for the treatment of neuronal and neurological damage associated with or caused by spinal cord injury, a method for minimizing neuronal and neurological damage associated with spinal cord injury, a method for reducing, limiting, and/or delaying scarring at the site of a spinal cord injury, a method for reducing or limiting the development of spontaneously occurring, post-operative or post-traumatic injury spinal adhesions, a method for educing or limiting the development of spontaneously occurring, post-operative or post-traumatic injury spinal tethering and a method for reducing or limiting the pain associated with arachnoiditis. In a preferred aspect of the disclosed methods, the defibrinogenating agent is ancrod.

### CROSS REFERENCE TO RELATED APPLICATIONS

This application claims priority from U.S. Provisional Application Serial Nos. 60/383,321 filed May 24, 2002 and No. 60/423,957 filed Nov. 4, 2002. The entire content of each of the previous applications is incorporated herein by reference.

### FIELD OF THE INVENTION

The present invention relates to the treatment of neuronal and neurological damage associated with spinal cord injury.

### BACKGROUND OF THE INVENTION

It has already been established that fibrin, a protein that helps form blood clots, may be involved in regulating the repair of nerve damage. Defibrinogenating agents, which function to reduce or remove circulating fibrinogen, which converts to fibrin at the site of neuronal damage, as well as fibrinolytic agents, which act directly to deplete fibrin, represent a new strategy for minimizing or repairing damage to the nervous system resulting from spinal cord injury and, potentially, other degenerative neurological diseases, such as, for example, multiple sclerosis.

Previous research has shown that fibrin accumulates in the lesions of people with multiple sclerosis, but until recently, the protein's role in nerve repair has not been studied extensively. In nerves outside the brain or spinal cord, loss of myelin can also occur after an injury. The body usually makes fibrin to allow blood to clot over a wound. However, researchers have previously also observed that it accumulates in damaged nerves immediately following the injury, and may play a key role in scar formation at the injury site. Such fibrin deposition is subsequently cleared, and this clearance seems to correlate with the timing of the regrowth and repair of peripheral nerves.

After a nerve injury, fibrinogen can leak from the blood into the damaged nerve. Once in the nerve, fibrinogen is converted into fibrin. While this is going on, myelin-repairing Schwann cells begin proliferating. The presence of fibrin, however, keeps the Schwann cells from beginning the repair process. It is only as fibrin is gradually dissolved that Schwann cells are able to begin the process of repairing the damaged myelin and regenerating the myelin sheath as the nerve grows.

A recent study directed to nerve regeneration after peripheral nerve damage, utilized Abbott Laboratories' brand of the defibrinogenating agent ancrod (VIPRINEX®, now under license to Empire Pharmaceuticals, New York, USA), a biological derived from the venom of the Malayan pit viper, to pharmacologically induce fibrin depletion in mice having crushed sciatic nerves, by decreasing the amount of fibrinogen available to convert to fibrin at the site of injury. The results of the study established that fibrin significantly slows the repair of damaged myelin in peripheral nerves.

It is believed that fibrin normally plays a role in keeping sheath cells in an immature state in which they are not capable of regenerating an intact myelin sheath. In the absence of fibrin, however, sheath cells are able to mature more quickly and are thus able to more efficiently remyelinate damaged nerves.

The study demonstrated that using a defibrinogenating agent to in turn limit the level of fibrin at the injury site, significantly enhanced the number of myelinating axons and prevented deposition of excess fibrin at the site. In the control group, where ancrod was not administered, fibrin deposition occurred and was shown to be an inhibitory factor in the early phase of remyelination. Akassoglou, K. et al., “Fibrin Inhibits Peripheral Nerve Remyelination by Regulating Schwann Cell Differentiation,” Neuron, 33:861-875 (2002).

Another recent study suggests that recovery from severe spinal cord injury may be enhanced by limiting the scarring process that generally follows such an injury. In this study, it was demonstrated in mice that without the physical barrier of scar tissue to impede the regeneration process, neurons on both sides of the injury site were able to grow and reestablish connections with each other over a period of two to three weeks, leading to substantial recovery of function. Heber-Katz, E. and Aglo, E., “Recovery in Spinal Cord Injury in Mice when Scarring is Minimized,” J Neuroscience Res., 67:2 (2002).

Fibrotic scarring may also be associated with post-operative and post-traumatic spinal adhesions and post-operative tethering (a process by which the spinal cord binds to the overlying dura). Moreover, spinal adhesions and tethering are not always a result of spinal trauma due to injury or surgery, but can also arise spontaneously.

In addition to scarring, other complications may also result from trauma to the spine, whether through injury or surgery. Arachnoiditis, for example, often develops following spinal trauma. Arachnoiditis is s very painful condition wherein the arachnoidea covering the spinal cord becomes inflamed as a result of, for example, meningitis or other disease process or trauma.

The arachnoidea is an extremely delicate membrane that is interposed between the much tougher and fibrous outer dura membrane (dura mater) and the innermost pia mater, which consists of collagenous fibers. The arachnoid membrane is separated from the pia mater by the subarachnoid space.

Applicant has now discovered that preventing fibrin deposition represents an effective means to enhance the nervous system's natural regenerative capacities. By limiting, removing or reducing fibrinogen, which converts to fibrin at the damaged site, inhibiting regeneration and initiating scarring, and consequently, reducing, limiting and/or delaying scarring at the injury site, defibrinogenating agents are therefore of benefit as a treatment for the repair of neuronal and neurological damage associated with spinal cord injuries. By acting directly to deplete fibrin, fibrinolytic agents are correspondingly effective as a treatment for the repair of neuronal and neurological damage associated with spinal cord injury.

In addition, systemic or local administration of defibrinogenating agents can reduce the frequency of spinal adhesions and tethering by interfering with the formation of fibrin and thereby limiting fibrin deposition and scarring at the surgical or otherwise traumatized site of the spinal cord.

### SUMMARY OF THE INVENTION

In accordance with the principles of the invention, Applicant has discovered that the administration of a therapeutically effective amount of a defibrinogenating agent, such as, for example, ancrod, urokinase, streptokinase and anticonvulsants such as, for example, phenobarbital or valproic acid, provides effective treatment of spinal cord injury.

### DETAILED DESCRIPTION OF THE INVENTION

In a preferred aspect of the invention, the defibrinogenating agent is ancrod, available, for example, under the trade names ARVIN® or VIPRINEX® (Empire Pharmaceuticals, Inc., New York, USA, under license from Abbott Laboratories previously, Knoll Pharmaceuticals/BASF).

As used herein, the term “ancrod” encompasses not only products prepared from the ancrod protease isolated from snake venom, but also any products containing ancrod proteins obtained through genetic manipulation.

Methods for the preparation of ancrod from snake venom are well known, and include, but are in no way limited to, the methods taught in U.S. Pat. No. 6,200,791 (licensed to Empire Pharmaceuticals, New York, USA), U.S. Pat. Nos. 3,743,722 and 3,879,369; Great Britain patent documents 1,094,301, 1,177,506 and 1,293,793; and German patent documents 2,428,955 and 2,734,427. Methods for the preparation of ancrod products through genetic manipulation are taught, for example, in U.S. Pat. No. 6,015,685 (licensed to Empire Pharmaceuticals, New York, USA).

As a further aspect of the invention, Applicant has also discovered that fibrinolytic agents such as tissue-plasminogen activator (tPA), recombinant tissue-plasminogen activator (rt-PA), fibrinolytic derivatives of recombinant tissue-plasminogen activator, such as, for example, reteplase (rPA), lanoteplase (nPA) and tenecteplase (TNK-tPA), as well as various advance-generation fibrates, for example, hypocholesterolemic drugs, such as, for example, fenofibrate, also effectively treat spinal cord injury.

In yet further aspects of the invention, the aforementioned defibrinogenating agents and fibrinolytic agents are also effective for minimizing neuronal and neurological damage associated with spinal cord injury, for aiding in the repair of neuronal and neurological damage associated with spinal cord injury and for reducing, limiting and/or delaying scarring at the site of a spinal cord injury.

According to the principles of the invention, the defibrinogenating or fibrinolytic agent may be administered in the form of a pharmaceutical medicament formulated for local or systemic administration. The pharmaceutical may, for example, be in the form of a gel, solution, powder or the like. Most preferably, however, the pharmaceutical will be in the form of a medicament formulated for intravenous administration.

Although the principals of the present invention have been described in detail by reference to various embodiments, it should be understood that this description is for illustration only, and the invention is not intended to be limited thereby. Various changes may be made, as the person of skill will recognize, without departing from the spirit and scope of the claims that follow.

We claim:

1. A method for the treatment of spinal cord injury, the method comprising administering to a patient in need of such treatment, a therapeutically effective amount of a defibrinogenating or fibrinolytic agent.

2. The method of claim 1, wherein the defibrinogenating agent is chosen from ancrod, urokinase, streptokinase or an anticonvulsant.

3. The method of claim 2, wherein the defibrinogenating agent is ancrod.

4. The method of claim 1, wherein the fibrinolytic agent is chosen from tissue-plasminogen activator (t-PA), recombinant tissue-plasminogen activator (rt-PA), fibrinolytic derivatives of recombinant tissue-plasminogen activator and fibrates, including hypocholesterolemic drugs.

5. The method of claim 4, wherein the fibrinolytic agent is chosen from reteplase (rPA), lanoteplase (nPA), tenecteplase (TNK-tPA) and fenofibrate.

6. A method for minimizing neuronal and neurological damage associated with spinal cord injury, the method comprising administering to a patient in need of such treatment, a therapeutically effective amount of a defibrinogenating or fibrinolytic agent.

7. The method of claim 6, wherein the defibrinogenating agent is chosen from ancrod, urokinase, streptokinase or an anticonvulsant.

8. The method of claim 7, wherein the defibrinogenating agent is ancrod.

9. The method of claim 6, wherein the fibrinolytic agent is chosen from tissue-plasminogen activator (t-PA), recombinant tissue-plasminogen activator (rt-PA), fibrinolytic derivatives of recombinant tissue-plasminogen activator and fibrates, including hypocholesterolemic drugs.

10. The method of claim 9, wherein the fibrinolytic agent is chosen from reteplase (rPA), lanoteplase (nPA), tenecteplase (TNK-tPA) and fenofibrate.

11. A method for aiding in the repair of neuronal and neurological damage associated with spinal cord injury, the method comprising administering to a patient in need of such treatment, a therapeutically effective amount of a defibrinogenating or fibrinolytic agent.

12. The method of claim 11, wherein the defibrinogenating agent is ancrod.

13. A method for minimizing neuronal and neurological damage associated with spinal cord injury, the method comprising administering to a patient in need of such treatment, a therapeutically effective amount of a defibrinogenating or fibrinolytic agent.

14. The method of claim 13, wherein the defibrinogenating agent is ancrod.

15. A method for aiding in the repair of neuronal and neurological damage associated with spinal cord injury, the method comprising administering to a patient in need of such treatment, a therapeutically effective amount of a defibrinogenating or fibrinolytic agent.

16. The method of claim 15, wherein the defibrinogenating agent is ancrod.

17. A method for reducing, limiting and/or delaying scarring at the site of a spinal cord injury, the method comprising administering to a patient in need of such treatment, a therapeutically effective amount of a defibrinogenating or fibrinolytic agent.

18. The method of claim 17, wherein the defibrinogenating agent is ancrod.

19. A method for reducing or limiting the development of spontaneously occurring, postoperative or post-traumatic injury spinal adhesions, the method comprising administering to a patient in need of such treatment, a therapeutically effective amount of a defibrinogenating or fibrinolytic agent.

20. The method of claim 19, wherein the defibrinogenating agent is ancrod.

21. A method for reducing or limiting the development of spontaneously occurring, postoperative or post-traumatic injury spinal tethering, the method comprising administering to a patient in need of such treatment, a therapeutically effective amount of a defibrinogenating or fibrinolytic agent.

22. The method of claim 21, wherein the defibrinogenating agent is ancrod.

23. A method for reducing or limiting the pain associated with arachnoiditis, the method comprising administering to a patient in need of such treatment, a therapeutically effective amount of a defibrinogenating or fibrinolytic agent.

24. The method of claim 23, wherein the defibrinogenating agent is ancrod.

25. The method of claim 1, wherein the defibrinogenating or fibrinolytic agent is in the form of a pharmaceutical medicament for systemic administration.

26. The method of claim 25, wherein the pharmaceutical medicament is administered intravenously.

27. The method of claim 1, wherein the defibrinogenating or fibrinolytic agent is in the form of a pharmaceutical medicament for local administration.

28. The method of claim 27, wherein the pharmaceutical medicament is administered as a gel, solution or powder.
